(19) United States
(12) Patent Application Publication
(10) Pub. No.: US 2006/0194998 A1
(43) Pub. Date: Aug. 31, 2006

(54) Process for making high octane gasoline with reduced benzene content

(76) Inventors: Benjamin S. Umansky, Fairfax, VA (US); Michael C. Clark, Pasadena, TX (US); Carlos N. Lopez, Seabrook, TX (US); John W. Viets, Fairfax, VA (US); C. Morris Smith, West University, TX (US); John H. Thurtell, Houston, TX (US); Tomas R. Melli, Haymarket, VA (US); Sean C. Smyth, Ashburn, VA (US)

Correspondence Address: ExxonMobil Research and Engineering Company, P. O. Box 900, Annandale, NJ, 08801-0900, US

(21) Appl. No.: 11/362,256

(22) Filed: Feb. 27, 2006

Related U.S. Application Data
(60) Provisional application No. 60/656,955, filed on Feb. 28, 2005.

(51) Int. Cl. C07C 2/68

(52) U.S. Cl. 585/467

(57) ABSTRACT

Solid phosphoric acid (SPA) olefin oligomerization process units may be converted to operation with a more environmentally favorable solid catalyst. The SPA units in which a light olefin feed is oligomerized to form gasoline boiling range hydrocarbon product, is converted unit to operation with a molecular sieve based olefin oligomerization catalyst comprising an MWW zeolite material. Besides being more environmentally favorable in use, the MWW based zeolites offer advantages in catalyst cycle life, selectivity. After loading of the catalyst, the converted unit is operated as a fixed-bed unit by passing a C₂- C₄ olefinic feed and a light aromatic co-feed containing benzene to a fixed bed of the MWW zeolite catalyst to effect alkylation of the benzene with the aromatic co-feed, typically at a temperature from 150 to 350° C., a pressure not greater than 7000 kpa, usually less than 4000 kPa and an olefin space velocity up to 10 WHSV.

### CROSS REFERENCE TO RELATED APPLICATIONS

This application claims priority from U.S. Application Ser. No. 60/656,955, filed 28 Feb. 2005entitled “Process for Making High Octane Gasolline with Reduced Benzene Content”.

This application is related to co-pending applications Ser. Nos.______, ,______ and______ , of even date, claiming priority, respectively from applications Ser. Nos. 60/656,954, 60/656,945, 60/656,946 and 60/656,947, all filed 28 Feb. 2005 and entitled respectively, “Gasoline Production By Olefin Polymerization”, “Vapor Phase Aromatics Alkylation Process”, “Liquid Phase Aromatics Alkylation Process” and “Olefins Upgrading Process”.

### FIELD OF THE INVENTION

This invention rleates to a process for the production of gasoline boiling range motor fuel by the polymerization of light olefins and their reaction with other hydrocarbons produced in the refining of petroluem crudes.

### BACKGROUND OF THE INVENTION

Following the introduction of catalytic cracking processes in petroleum refining in the early 1930s, large amounts of olefins, particularly light olefins such as ethylene, propylene, butylene, became available in copious quantities from catalytic cracking plants in refineries. While these olefins may be used as petrochemical feedstock, many conventional petroleum refineries producing petroleum fuels and lubricants are not capable of diverting these materials to petrochemical uses. Processes for producing fuels from these cracking off gases are therefore desirable and from the early days, a number of different processes evolved. The early thermal polymerization process was rapidly displaced by the superior catalytic processes of which there was a number. The first catalytic polymerization process used a sulfuric acid catalyst to polymerize isobutene selectively to dimers which could then be hydrogenated to produce a branched chain octane for blending into aviation fuels. Other processes polymerized isobutylene with normal butylene to form a co-dimer which again results in a high octane, branched chain product. An alternative process uses phosphoric acid as the catalyst, on a solid support and this process can be operated to convert all the C₃ and C₄ olefins into high octane rating, branched chain polymers. This process may also operate with a C₄ olefin feed so as to selectively convert only isobutene or both n-butene and isobutene. This process has the advantage over the sulfuric acid process in that propylene may be polymerized as well as the butenes and at the present time, the solid phosphoric acid [SPA] polymerization process remains the most important refinery polymerization process for the production of motor gasoline.

In the SPA polymerization process, feeds are pretreated to remove hydrogen sulfide and mercaptans which would otherwise enter the product and be unacceptable, both from the view point of the effect on octane and upon the ability of the product to conform to environmental regulations. Typically, a feed is washed with caustic to remove hydrogen sulfide and mercaptans, after which it is washed with water to remove organic basis and any caustic carryover. Because oxygen promotes the deposition of tarry materials on the catalyst, both the feed and wash water are maintained at a low oxygen level. Additional pre-treatments may also be used, depending upon the presence of various contaminants in the feeds. With the most common solid phosphoric acid catalyst, namely phosphoric acid on kieselguhr, the water content of the feed needs to be controlled carefully because although a limited water content is required for catalyst activity, the catalyst softens in the presence of excess water so that the reactor may plug with a solid, stone-like material which is difficult to remove without drilling or other arduous operations. Conversely, if the feed is too dry, coke tends to deposit on the catalyst, reducing its activity and increasing the pressure drop across the reactor. As noted by Henckstebeck, the distribution of water between the catalyst and the reactants is a function of temperature and pressure which vary from unit to unit, and for this reason different water concentrations are required in the feeds to different units. Petroleum Processing Principles And Applications, R. J. Hencksterbeck McGraw-Hill, 1959.

For the production of motor gasoline only butene and lighter olefins are employed as feeds to polymerization processes as heavier olefins up to about C₁₀ or C₁₁ can be directly incorporated into the gasoline. With the PSA process, propylene and butylene are satisfactory feedstocks and ethylene may also be included, to produce a copolymer product in the gasoline boiling range. Limited amounts of butadiene may be permissible although this diolefin is undesirable because of its tendency to produce higher molecular weight polymers and to accelerate deposition of coke on the catalyst. The process generally operates under relatively mild conditions, typically between 150° and 200° C., usually at the lower end of this range between 150° and 180° C., when all butenes are polymerized. Higher temperatures may be used when propylene is included in the feed. In a well established commercial SPA polymerization process, the olefin feed together with paraffinic diluent, is fed to the reactor after being preheated by exchange with the reaction effluent.

There are two general types of units used for the SPA process, based on the reactor type, the unit may be classified as having chamber reactors or tubular reactors. The chamber reactor contains a series of catalyst beds with bed volume increasing from the inlet to the outlet of the reactor, with the most common commercial design having five beds. The catalyst load distribution is designed to control the heat of conversion.

Chamber reactors usually operate with high recycle rates. The recycle stream, depleted in olefin content following polymerization, is used to dilute the olefin at the inlet of the reactor and to quench the inlets of the following beds. Chamber reactors usually operate at pressure of approximately 3500-5500 kPag (about 500-800 psig) and temperature between 180° to 200° C. (about 350°- 400° F.). The conversion, per pass of the unit, is determined by the olefin specification in the LPG product stream. Fresh feed LHSV is usually low, approximately 0.4 to 0.8 hr⁻¹. The cycle length for chamber reactors is typically between 2 to 4 months.

The tubular reactor is basically a shell-and-tube heat exchanger in which the polymerization reactions take place in a number of parallel tubes immersed in a cooling medium and filled with the SPA catalyst. Reactor temperature is controlled with the cooling medium, invariably water in commercial units, that is fed on the shell side of the reactor. The heat released from the reactions taking place inside the tubes evaporates the water on the shell side. Temperature profile in a tubular reactor is close to isothermal. Reactor temperature is primarily controlled by means of the shell side water pressure (controls temperature of evaporation) and secondly by the reactor feed temperature. Tubular reactors usually operate at pressure between 5500 and 7500 kPag (800-1100 psig) and temperature of around 200° C. (about 400° F.). Conversion per pass is usually high, around 90 to 93% and the overall conversion is around 95 to 97%. The space velocity in tubular reactors is typically high, e.g., 2 to 3.5 hr⁻¹ LHSV. Cycle length in tubular reactors is normally between 2 to 8 weeks.

Another problem facing the refining industry at the present is that current refinery regulations related to motor fuels have limited the amount of benzene which is permissible in motor fuels. These regulations have produced substantial changes in refinery operation. To comply with these regulations, some refineries have excluded C₆ compounds from reformer feed so as to avoid the production of benzene directly. An alternative approach is to remove the benzene from the reformate after it is formed by means of an aromatics extraction process such as the Sullfolane Process or UDEX Process. Well-integrated refineries with aromatics extraction units have flexibility to accommodate the benzene requirements but it is more difficult to meet the benzene specification for refineries without the aromatic extraction units.

The removal of benzene is, however, accompanied by a decrease in product octane quality since benzene and other single ring aromatics make a positive contribution to product octane. Certain processes have been proposed for converting the benzene in aromatics-containing refinery streams to the less toxic alkylaromatics such as toluene and ethyl benzene which themselves are desirable as high octane blend components. One process of this type was the Mobil Benzene Reduction (MBR) Process which, like the closely related MOG Process, used a fluidized zeolite catalyst in a riser reactor to alkylate benzene in reformate to from alkylaromatics such as toluene. The MBR and MOG processes are described in U.S. Pat. Nos. 4,827,069; 4,950,387; 4,992,607 and 4,746,762.

The fluid bed MBR Process uses a shape selective, metallosilicate catalyst, preferably ZSM-5, to convert benzene to alkylaromatics using olefins from sources such as FCC or coker fuel gas, excess LPG or light FCC naphtha. Normally, the MBR Process has relied upon light olefin as alkylating agent for benzene to produce alkylaromatics, principally in the C₇-C₈ range. Benzene is converted, and light olefin is also upgraded to gasoline concurrent with an increase in octane value. Conversion of light FCC naphtha olefins also leads to substantial reduction of gasoline olefin content and vapor pressure. The yield-octane uplift of MBR makes it one of the few gasoline reformulation processes that is actually economically beneficial in petroleum refining.

Like the MOG Process, however, the MBR Process required considerable capital expenditure, a factor which did not favor its widespread application in times of tight refining margins. The MBR process also used higher temperatures and C₅+ yields and octane ratings could in certain cases be deleteriously affected another factor which did not favor widespread utilization. Other refinery processes have also been proposed to deal with the problems of excess refinery olefins and gasoline; processes of this kind have often functioned by the alkylation of benzene with olefins or other alkylating agents such as methanol to form less toxic alkylaromatic precursors. Exemplary processes of this kind are described in U.S. Pat. Nos. 4,950,823; 4,975,179; 5,414,172; 5,545,788; 5,336,820; 5,491,270 and 5,865,986.

While these known processes are technically attractive they, like the MOG and MBR processes, have encountered the disadvantage of needing to a greater or lesser degree, some capital expenditure, a factor which militates strongly against them in present circumstances. What is needed is a process that is, as near as possible, a “drop-in” replacement for and existing refinery process, capable of utilizing existing refinery equipment as far as possible.

For these reasons, a refinery process able to alkylate benzene (or other aromatics) with the olefins would be beneficial not only to meet benzene specification but also to increase motor fuel volume with high-octane alkylaromatic compounds. For some refineries, the reactive removal of C₂/C₃ olefins could alleviate fuel gas capacity limitations. Such a process should:
Upgrade C₂ and C3 olefin from fuel gas to high octane blending gasoline
Increase flexibility in refinery operation to control benzene content in the gasoline blending pool
Allow refineries with benzene problems to feed the C₆ components (low blending octane values) to the reformer, increasing both the hydrogen production from the reformer and the blend pool octane. Benzene produced in the reformer will be removed in order to comply with gasoline product specifications.

Have the potential, by the removal of olefins from the fuel gas, to increase capacity in the fuel system facility. For some refineries this benefit could allow an increase in severity in some key refinery process, FCC, hydrocracker, coker, etc.

In distinction to similar processes now current for chemicals production which require high purity feed components, allow normal refinery streams with their concomitant levels of impurities to be used, at consequent lower cost.

Co-pending U.S. patent application Ser. No. ______, claiming priority of application Ser. No. 60/656,954 describes a process for the conversion of light olefins such as ethylene, propylene, and butylene to gasoline boiling range motor fuels using a solid polymerization (condensation, oligomerization) catalyst which is capable of being used as a replacement for solid phosphoric acid catalyst in process units which have previously been used for the SPA process. The catalyst described in the application is a solid, particulate catalyst which is non-corrosive, which is stable in fixed bed operation, which exhibits the capability of extended cycle duration before regeneration is necessary and which can be readily handled and which can be finally disposed of simply and economically without encountering significant environmental problems. Thus, this process provides an economically attractive alternative to the established SPA process which provides a solution to the problem of using the light olefin production in an economic manner. Thus, the process described in U.S. application Ser. No. (claiming priority of Ser. No. 60/656,954) can be characterized as a near “drop-in” replacement for the well-established SPA Process, being readily capable of operation within the process units used for the known process.

### SUMMARY OF THE INVENTION

We have now devised a process which enables light refinery olefins to be readily converted to gasoline boiling range fuel products and, at the same time, enables the refinery to comply with gasoline benzene specifications. The process is similar to the process described in U.S. Application Ser. No.______ (claiming priority of Ser. No. 60/656,954) in that light refinery olefins are converted to higher boiling products in the gasoline boiling range in a fixed bed catalytic process using a zeolite catalyst, the difference being that in the present case, the reactions are carried out in the presence of benzene and optionally other light aromatic compounds, to produce a product possessing a high octane rating characteristic of the alkylaromatics resulting from the alkylation of the benzene with the olefins present in the feed.

According to the present invention, a mixed light olefin feed such as a mix of at least two of ethylene, propylene, and butylene, optionally with other light olefins, are reacted in the presence of a light aromatic compound such as benzene or a single ring aromatic with a short chain alkyl side chain to form a gasoline boiling range [C₅+-200° C.] [C₅+- 400° F.] product containing akylaromatics. The reaction is carried out in the presence of a catalyst which comprises a member of the MWW family of zeolites, a family which is currently known to includes zeolite PSH 3, MCM-22, MCM-36, MCM-49, MCM-56, SSZ 25, ERB-1 and ITQ-1. The process is carried out as fixed bed operation; the reactor may be either of the chamber type with feed dilution or added quench to control the heat release or in a tubular reactor with external cooling.

In additional to their easy handling and amenability to regeneration, the solid catalysts used in the present process exhibit better activity, selectivity and stability than solid phosphoric acid; compared to SPA, MCM-22 itself is at least five times more active and significantly more stable for the production of motor gasoline by the polymerization of light olefin feeds. The catalytic performance of regenerated MCM-22 catalyst is comparable to that of the fresh MCM-22 catalyst, demonstrating that the catalyst is amenable to conventional oxidative regeneration techniques.

The conversion of an SPA process unit to operation with the present molecular sieve based catalysts therefore comprises, in principle, withdrawing the solid phosphoric acid [SPA] catalyst from the unit and loading an olefin condensation catalyst comprising an MWW zeolite material into the reactor of the process unit. Following unit conversion, the refinery olefins may be processed with the light aromatic stream to form the high octane, low benzene gasoline fuel product using the appropriate process conditions described below.

### DRAWINGS

FIG. 1 shows a process schematic for the olefin polymerization unit for converting light refinery olefins and benzene to motor gasoline by the present process.

FIG. 2 is a graph showing the alkylation of a light aromatic fraction with ethylene and/or propylene.

### DETAILED DESCRIPTION OF THE INVENTION

SPA Unit Conversion

The present process is for the upgrading of light olefins in the presence of light aromatics to produce a high octane rating, aromatic motor gasoline of reduced benzene content which may be fed directly into the refinery gasoline pool as a high value blend component. Also, by suitable adjustment of the reaction conditions, other fuels, for example, aromatic road diesel and kerojet blend stocks may be produced. The process is intended to provide a replacement for the SPA polymerization process with the added advantage of removing benzene from the refinery gasoline blend pool. The present upgrading process uses a catalyst which can be used as a direct replacement for SPA and so enables existing SPA units to be used directly with the new catalyst with minimal unit modification, in this way, the advantages of the new catalyst and process can be effectively exploited while retaining the economic benefit of most existing refinery equipment.

The process units used for the operation of the SPA process for the catalytic condensation of light olefins to produce motor fuels are well known. The chamber type units typically comprise a feed surge drum to which the olefins and any diluent are supplied, followed by a heat exchanger in which the feed is preheated by exchange with the reactor effluent, after which it is charged to the reactor where the polymerization (condensation) takes place. Control of the heat release in the chamber reactor is accomplished both by feed dilution and by the injection of recycled quench between catalyst beds in the reactor. The reactor effluent, cooled in exchange with the feed, is directed to a flash drum where the flash vapor is condensed and the condensate cooled. Some of the condensate is recycled for use as feed diluent and quench. Flash drum liquid flows to a stabilizer where the polymer gasoline product at the desired Reid Vapor Pressure [RVP] and light ends are separated. The light ends may be sent to a C₃-C₄ splitter depending on the refinery needs. Units with tubular reactors have similar ancillary units except that in this case, the necessity for the recycle and quench equipment is removed.

Like SPA, the molecular sieve catalysts used in the present process are non-corrosive but possess significant advantages with respect to SPA, in that they are more stable, less subject to break down and are largely unaffected by the amount of water in the feed. The present catalysts are readily regenerable using conventional hydrogen stripping or oxidative regeneration, after which complete catalytic activity is substantially restored. Cycle times before regeneration or reactivation is required may be six months, one year, or even longer, representing a significant improvement over SPA. Since conventional SPA condensation units necessarily include facilities for the discharge and reloading of catalysts as a result of the short life of a catalyst, these units may readily accommodate the present molecular sieve catalysts. The SPA units do not, however, include facilities for in-situ regeneration since the SPA catalyst is used on a once-through basis before it requires disposal. The molecular sieve catalysts used in the present process, however, are fully regenerable and for this purpose, can be withdrawn from the reactors for ex-situ regeneration. This will typically be a simple matter to arrange using the spent catalyst discharge equipment of the SPA unit. Similarly, the SPA charging equipment lends itself directly to the charging of the zeolite catalysts into the reactors. If suitable provision for in situ regeneration can be made, an appropriate regeneration circuit may be present in the unit.

Unit Conversion

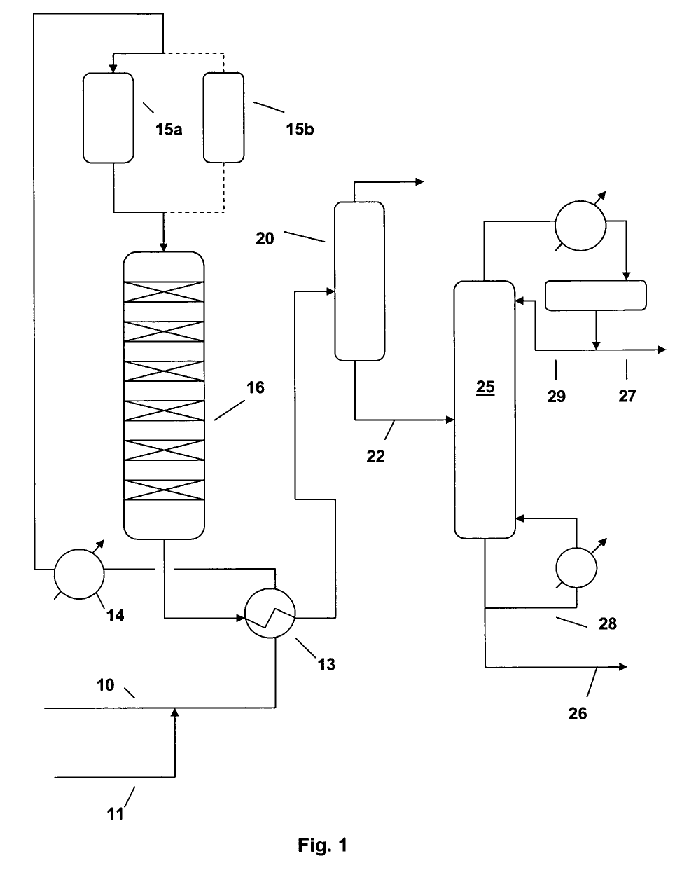

A schematic for a converted olefin condensation unit made by the conversion of an existing SPA unit (chamber type) is shown in simplified form in FIG. 1. A light olefin feed, typically C₂, C₃ or C₄ olefins or mixtures of these olefins from an FCC gas plant, is led into the unit through line 10 and combined with a light aromatic stream containing benzene entering through line 11. The combined stream then passes through heat exchanger 13 in which it picks up heat from the reactor effluent before being brought to reaction temperature in heater 14. From heater 14, the feed flows through a guard bed reactor 15a to remove contaminants such as organic nitrogen and sulfur-containing impurities. The guard bed may be operated on the swing cycle with two beds, 15a, 15b, one bed being used on stream for contaminant removal and the other on regeneration in the conventional manner. If desired, a three-bed guard bed system may be used with the two beds used in series for contaminant removal and the third bed on regeneration. With a three guard system used to achieve low contaminant levels by the two-stage series sorption, the beds will pass sequentially through a three-step cycle of: regeneration, second bed sorption, first bed sorption. The mixed olefin/benzene charge then passes through reactor 16 in which the olefin is reacted with the aromatics to form the desired alkylaromatic product of reduced benzene content during its passage over a sequence of catalyst beds in the reactor. Effluent passes out of the reactor through heat exchanger 13 and then to flash drum 20 The alkylaromatic product passes out of flash drum 20 through line 22 to the fractionator 25 to provide the final stabilized gasoline blend component in line 26 with reboil loop 28 providing column heat; light ends including unreacted olefins pass out through line 27 from reflux loop 29.

Normally, the recycle and quench used in the chamber-type olefin polymerization units will not be necessary since the incoming aromatics stream provides adequate dilution of the olefin stream for temperature control. If, however, additional feed dilution and/or quench are required for temperature control for example, if it is desired to operate the unit without aromatic co-feed as a simple olefin polymerization unit as described in co-pending application No. (claiming priority of U.S. Ser. No. 60/656,954)(“Gasoline Production by Olefin Polymerization”) with chamber type reactors, provision for recycle and quench may be made as described in that application. The product recovery section of a converted tubular type SPA unit may be similar with the exception that the recycle and quench lines are not necessary in any event since any required reactor temperature control is effected by means of the liquid coolant on the shell side of the reactor assembly.

The catalyst used in the guard bed will normally be the same catalyst used in the alkylation reactor as a matter of operating convenience but this is not required: if desired another catalyst or sorbent to remove contaminants from the feed may used, typically a cheaper guard bed sorbent, e.g a used catalyst from another process or alumina. The objective of the guard bed is to remove the contaminants from the feed before the feed comes to the reaction catalyst and provided that this is achieved, there is wide variety of choice as to guard bed catalysts and conditions useful to this end.

Catalyst

The catalysts used in the present process contain, as their essential catalytic component, a molecular sieve of the MWW type. The MWW family of zeolite materials has achieved recognition as having a characteristic framework structure which presents unique and interesting catalytic properties. The MWW topology consists of two independent pore systems: a sinusoidal ten-member ring [10 MR] two dimensional channel separated from each other by a second, two dimensional pore system comprised of 12 MR super cages connected to each other through 10 MR windows. The crystal system of the MWW framework is hexagonal and the molecules diffuse along the [100] directions in the zeolite, i.e., there is no communication along the c direction between the pores. In the hexagonal plate-like crystals of the MWW type zeolites, the crystals are formed of relatively small number of units along the c direction as a result of which, much of the catalytic activity is due to active sites located on the external surface of the crystals in the form of the cup-shaped cavities. In the interior structure of certain members of the family such as MCM-22, the cup-shaped cavities combine together to form a supercage. The MCM-22 family of zeolites has attracted significant scientific attention since its initial announcement by Leonovicz et al. in Science 264, 1910-1913 [1994] and the later recognition that the family is currently known to include a number of zeolitic materials such as PSH 3, MCM-22, MCM 49, MCM 56, SSZ 25, ERB-1, ITQ-1, and others. Lobo et al. AlChE Annual Meeting 1999, Paper 292J.

The relationship between the various members of the MCM-22 family have been described in a number of publications. Three significant members of the family are MCM-22, MCM-36, MCM-49, and MCM-56. When initially synthesized from a mixture including sources of silica, alumina, sodium, and hexamethylene imine as an organic template, the initial product will be MCM-22 precursor or MCM-56, depending upon the silica: alumina ratio of the initial synthesis mixture. At silica:alumina ratios greater than 20, MCM-22 precursor comprising H-bonded vertically aligned layers is produced whereas randomly oriented, non-bonded layers of MC-56 are produced at lower silica:alumina ratios. Both these materials may be converted to a swollen material by the use of a pillaring agent and on calcination, this leads to the laminar, pillared structure of MCM-36. The as-synthesized MCM-22 precursor can be converted directly by calcination to MCM-22 which is identical to calcined MCM-49, an intermediate product obtained by the crystallization of the randomly oriented, as-synthesized MCM-56. In MCM-49, the layers are covalently bonded with an interlaminar spacing slightly greater than that found in the calcined MCM-22/MCM 49 materials. The unsynthesized MCM-56 may be calcined itself to form calcined MCM 56 which is distinct from calcined MCM-22/MCM-49 in having a randomly oriented rather than a laminar structure. In the patent literature MCM-22 is described in U.S. Pat. No. 4,954,325 as well as in U.S. 5,250,777; 5,284,643 and 5,382,742. MCM-49 is described in U.S. 5,236,575; MCM-36 in U.S. 5,229,341 and MCM-56 in U.S. 5,362,697.

The preferred zeolitic material for use in the catalyst of the present process is MCM-22 although zeolite MCM-49 may be found to have certain advantages relative to MCM-22. It has been found that the MCM-22 may be either used fresh, that is, not having been previously used as a catalyst or alternatively, regenerated MCM-22 may be used. Regenerated MCM-22 may be used after it has been used in any of the catalytic processes for which it is suitable, including the present process in which the catalyst has shown itself remain active after even multiple regenerations. It may also be possible to use MWW catalysts which have previously been used in other commercial processes and for which they are no longer acceptable, for example, MCM-22 catalyst which has previously been used for the production of aromatics such as ethylbenzene or cumene, normally using reactions such as alkylation and transalkylation. The cumene production (alkylation) process is described in U.S. Pat. No. US 4,992,606 (Kushnerick et al). Ethylbenzene production processes are described in U.S. Pat. Nos. 3,751,504 (Keown); 4,547,605 (Kresge); and 4,016,218 (Haag); U.S. Pat. Nos. 4,962,256; 4,992,606; 4,954,663; 5,001,295; and 5,043,501 describe alkylation of aromatic compounds with various alkylating agents over catalysts comprising MWW zeolites such as PSH-3 or MCM-22. U.S. Pat. No. 5,334,795 describes the liquid phase synthesis of ethylbenzene with MCM-22. As noted above, MCM-22 catalysts may be regenerated after catalytic use in these processes and other aromatics production processes by conventional air oxidation techniques similar to those used with other zeolite catalysts. Conventional air oxidation techniques are also suitable when regenerating the catalysts after use in the present process.

In addition to the MWW active component, the catalysts for use in the present process will often contain a matrix material or binder in order to give adequate strength to the catalyst as well as to provide the desired porosity characteristics in the catalyst. High activity catalysts may, however, be formulated in the binder-free form by the use of suitable extrusion techniques, for example, as described in U.S. Pat. No. 4,908,120. When used, matrix materials suitably include alumina, silica, silica alumina, titania, zirconia, and other inorganic oxide materials commonly used in the formulation of molecular sieve catalysts. For use in the present process, the level of MCM-22 in a finished matrixed catalyst will be typically from 20 to 70 % by weight, and in most cases from 25 to 65 % by weight. In manufacture of a matrixed catalyst, the active ingredient will typically be mulled with the matrix material using an aqueous suspension of the catalyst and matrix, after which the active component and the matrix are extruded into the desired shape, for example, cylinders, hollow cylinders, trilobe, quadlobe, etc. A binder material such as clay may be added during the mulling in order to facilitate extrusion, increase the strength of the final catalytic material and to confer other desirable solid state properties. The amount of clay will not normally exceed 10% by weight of the total finished catalyst. Self-bound catalysts (alternatively referred to as unbound or binder-free catalysts), that is, catalysts which do not contain a separately added matrix or binder material, are useful and may be produced by the extrusion method described in U.S. Pat. No. 4,582,815, to which reference is made for a description of the method and of the extruded products obtained by its use. The method described there enables extrudates having high constraining strength to be produced on conventional extrusion equipment and accordingly, the method is eminently suitable for producing the high activity self-bound catalysts. The catalysts are produced by mulling the zeolite, as described in U.S. Pat. No. 4,582,815, with water to a solids level of 25 to 75 wt% in the presence of 0.25 to 10 wt% of basic material such as sodium hydroxide. Further details are to be found in U.S. Pat. No. 4,582,815. Generally, the self-bound catalysts can be characterized as particulate catalysts in the form, for instance, of extrudates or pellets, containing at least 90 wt. pct., usually at least 95 wt. pct., of the active zeolite component with no separately added binder material e.g. alumina, silica-alumina, silica, titania, zirconia etc. Extrudates may be in the conventional shapes such as cylinders, hollow cylinders, trilobe, quadlobe, flat platelets etc.

As noted above, MCM-22 and other catalysts of this family may be regenerated after catalytic use for example, in the present process or in the cumene, ethylbenzene and other aromatics production processes, with the regeneration carried out by conventional air oxidation techniques similar to those used with other zeolite catalysts. Regeneration of the catalyst after use in the present process results in only a modest activity loss, with the catalyst maintaining more than 95% of fresh activity after the first regeneration. Even after multiple regenerations, a reasonable and acceptable level of activity is retained. The catalyst has been found to maintain more than 80 % of fresh activity after 6 regenerations. Following the air oxidation, the catalyst may be reconditioned by aqueous reconditioning treatment using water or a mildly alkaline solution, for example, a dilute solution of ammonia or sodium carbonate. Treatment with water alone at ambient temperatures has been found to be effective: the air-regenerated catalyst is cooled and then immersed in a water bath after which it is dried and returned to service. The reconditioning treatment may be continued for the empirically determined time which results in an improvement in catalyst properties. It is theorized that the reconditioning treatment enables the silanol groups on the surface of the zeolite to be re-formed after the regeneration treatment with a consequent restoration of catalytic properties which, in favorable cases, may provide a catalyst almost comparable to a fresh catalyst.

The catalyst used in the guard bed will normally be the same catalyst used in the alkylation reactor as a matter of operating convenience but this is not required: if desired another catalyst or sorbent to remove contaminants from the feed may used, typically a cheaper guard bed sorbent, e.g a used catalyst from another process or alumina. The objective of the guard bed is to remove the contaminants from the feed before the feed comes to the reaction catalyst and provided that this is achieved, there is wide variety of choice as to guard bed catalysts and conditions useful to this end. The volume of the guard bed will normally not exceed about 20% of the total catalyst bed volume of the unit.

Olefin Feed

The light olefins used as the feed for the present process are normally obtained by the catalytic cracking of petroleum feedstocks to produce gasoline as the major product. The catalytic cracking process, usually in the form of fluid catalytic cracking (FCC) is well established and, as is well known, produces large quantities of light olefins as well as olefinic gasolines and by-products such as cycle oil which are themselves subject to further refining operations. The olefins which are primarily useful in the present process are the lighter olefins from ethylene up to butene; although the heavier olefins may also be included in the processing, they can generally be incorporated directly into the gasoline product where they provide a valuable contribution to octane. The present process is highly advantageous in that it will operate readily not only with butene and propylene but also with ethylene and thus provides a valuable route for the conversion of this cracking by-product to the desired gasoline product. For this reason as well as their ready availability in large quantities in a refinery, mixed olefin streams such a FCC Off-Gas streams (typically containing ethylene, propylene and butenes) may be used. Conversion of the C₃ and C₄ olefin fractions from the cracking process provides a direct route to the branch chain C₆, C₇ and C₈ products which are so highly desirable in gasoline from the view point of boiling point and octane. Besides the FCC unit, the mixed olefin streams may be obtained from other process units including cokers, visbreakers and thermal crackers. The presence of diolefins which may be found in some of these streams is not disadvantageous since catalysis on the MWW family of zeolites takes place on surface sites rather than in the interior pore structure as with more conventional zeolites so that plugging of the pores is less problematic catalytically. Appropriate adjustment of the process conditions will enable co-condensation products to be produced when ethylene, normally less reactive than its immediate homologs, is included in the feed. The compositions of two typical FCC gas streams is given below in Tables 1 and 2, Table 1 showing a light FCC gas stream and Table 2 a stream from which the ethylene has been removed in the gas plant for use in the refinery fuel system.

TABLE 1
FCC Light Gas Stream

| Component | Wt. Pct. | Mol. Pct. |
|---|---|---|
| Ethane | 3.3 | 5.1 |
| Ethylene | 0.7 | 1.2 |
| Propane | 14.5 | 15.3 |
| Propylene | 42.5 | 46.8 |
| Iso-butane | 12.9 | 10.3 |
| n-Butane | 3.3 | 2.6 |
| Butenes | 22.1 | 18.32 |
| Pentanes | 0.7 | 0.4 |

TABLE 2
C₃-C₄ FCC Gas Stream

| Component | Wt. Pct. |
|---|---|
| 1-Propene | 18.7 |
| Propane | 18.1 |
| Isobutane | 19.7 |
| 2-Me-1-propene | 2.1 |
| 1-Butene | 8.1 |
| n-Butane | 15.1 |
| Trans-2-butene | 8.7 |
| Cis-2-butene | 6.5 |
| Isopentane | 1.5 |
| C3 Olefins | 18.7 |
| C4 Olefins | 25.6 |
| Total Olefins | 44.3 |

While the catalysts used in the present process are robust they do have sensitivity to certain contaminants (the conventional zeolite deactivators), especially organic compounds with basic nitrogen as well as sulfur-containing organics. It is therefore preferred to remove these materials prior to entering the unit if extended catalyst life is to be expected. Scrubbing with contaminant removal washes such as caustic, MEA or other amines or aqueous wash liquids will normally reduce the sulfur level to an acceptable level of about 10-20 ppmw and the nitrogen to trace levels at which it can be readily tolerated. One attractive feature about the present process is that it is not unduly sensitive to water, making it less necessary to control water entering the reactor than it is in SPA units. Unlike SPA, the zeolite catalyst does not require the presence of water in order to maintain activity and therefore the feed may be dried before entering the unit. In conventional SPA units, the water content typically needs to be held between 300 to 500 ppmw for adequate activity while, at the same time, retaining catalyst integrity. The present zeolite catalysts, however, may readily tolerate up to about 1,000 ppmw water although levels above about 800 ppmw may reduce activity, depending on temperature.

Aromatic Feed

In addition to the light olefin feed, an aromatic stream containing benzene is fed into the process, as described above. This stream may contain other single ring aromatic compounds including alkylaromatics such as toluene, ethylbenzene, propylbenzene (cumene) and the xylenes. In refineries with associated petrochemical capability, these alkylaromatics will normally be removed for higher value use as chemicals or, alternatively, may be sold separately for such uses. Since they are already considered less toxic than benzene, there is no environmental requirement for their inclusion in the aromatic feed stream but, equally, there is no prejudice against their presence unless conditions lead to the generation of higher alkylaromatics which fall outside the gasoline range or which are undesirable in gasoline, for example, durene. The amount of benzene in this stream is governed mainly by its source and processing history but in most cases will typically contain at least about 5 vol. % benzene, although a minimum of 12 vol. % is more typical, more specifically about 20 vol. % to 60 vol. % benzene. Normally, the main source of this stream will be a stream from the reformer which is a ready source of light aromatics. Reformate streams may be full range reformates, light cut reformates, heavy reformates or heart cut reformates. These fractions typically contain smaller amounts of lighter hydrocarbons, typically less than about 10% C₅ and lower hydrocarbons and small amounts of heavier hydrocarbons, typically less than about 15% C₇+hydrocarbons. These reformate feeds usually contain very low amounts of sulfur as, usually, they have been subjected to desulfurization prior to reforming so that the resulting gasoline product formed in the present process contains an acceptably low level of sulfur for compliance with current sulfur specifications.

Reformate streams will typically come from a fixed bed, swing bed or moving bed reformer. The most useful reformate fraction is a heart-cut reformate. This is preferably reformate having a narrow boiling range, i.e. a C₆ or C6/C₇ fraction. This fraction is a complex mixture of hydrocarbons recovered as the overhead of a dehexanizer column downstream from a depentanizer column. The composition will vary over a range depending upon a number of factors including the severity of operation in the reformer and the composition of the reformer feed. These streams will usually have the C5, C₄ and lower hydrocarbons removed in the depentanizer and debutanizer. Therefore, usually, the heart-cut reformate will contain at least 70 wt. % C6 hydrocarbons, and preferably at least 90 wt. % C₆ hydrocarbons.

Other sources of aromatic, benzene-rich feeds include a light FCC naphtha, coker naphtha or pyrolysis gasoline but such other sources of aromatics will be less important or significant in normal refinery operation.

By boiling range, these benzene-rich fractions can normally be characterized by an end boiling point of about 120° C. (250° F.)., and preferably no higher than about 110° C. (230° F.). Preferably, the boiling range falls between 40° and 100° C. (100° F. and 212° F.)., and more preferably between the range of 65° to 95° C. (150° F. to 200° F.) and even more preferably within the range of 70° to 95° C. (160° F. to 200° F.).

The compositions of two typical heart cut reformate streams are given in Tables 3 and 4 below. The reformate shown in Table 4 is a relatively more paraffinic cut but one which nevertheless contains more benzene than the cut of Table 3, making it a very suitable substrate for the present alkylation process.

TABLE 3
C6-C7 Heart Cut Reformate

| | |
|---|---|
| RON | 82.6 |
| MON | 77.3 |
| Composition, wt. pct. | |
| i-C₅ | 0.9 |
| n-C₅ | 1.3 |
| C₅ napthenes | 1.5 |
| i-C₆ | 22.6 |
| n-C₆ | 11.2 |
| C₆ naphthenes | 1.1 |
| Benzene | 32.0 |
| i-C₇ | 8.4 |
| n-C₇ | 2.1 |
| C₇ naphthenes | 0.4 |
| Toluene | 17.7 |
| i-C₈ | 0.4 |
| n-C₈ | 0.0 |
| C₈ aromatics | 0.4 |

TABLE 4
Paraffinic C6-C7 Heart Cut Reformate

| | |
|---|---|
| RON | 78.5 |
| MON | 74.0 |
| Composition, wt. pct. | |
| i-C₅ | 1.0 |
| n-C₅ | 1.6 |
| C₅ napthenes | 1.8 |
| i-C₆ | 28.6 |
| n-C₆ | 14.4 |
| C₆ naphthenes | 1.4 |
| Benzene | 39.3 |
| i-C₇ | 8.5 |
| n-C₇ | 0.9 |
| C₇ naphthenes | 0.3 |
| Toluene | 2.3 |

Reformate streams will come from a fixed bed, swing bed or moving bed reformer. The most useful reformate fraction is a heart-cut reformate. This is preferably reformate having a narrow boiling range, i.e. a C₆ or C₆/C₇ fraction. This fraction is a complex mixture of hydrocarbons recovered as the overhead of a dehexanizer column downstream from a depentanizer column. The composition will vary over a range depending upon a number of factors including the severity of operation in the reformer and the composition of the reformer feed. These streams will usually have the C₅, C₄ and lower hydrocarbons removed in the depentanizer and debutanizer. Therefore, usually, the heart-cut reformate may contain at least 70 wt. % C₆ hydrocarbons (aromatic and non-aromatic), and preferably at least 90 wt. % C₆ hydrocarbons.

Other sources of aromatic, benzene-rich feeds include a light FCC naphtha, coker naphtha or pyrolysis gasoline but such other sources of aromatics will be less important or significant in normal refinery operation.

Product Formation

During the process, a number of mechanistically different reactions take place. The principle reactions taking place will be alkylation and transalkylation reactions between the aromatics and the olefins. These reactions will predominate significantly over the minor degree of olefin oligomerization which occurs since the aromatics are readily sorbed onto the catalyst and preferentially occupy the catalytic sites making olefin self-condensation reactions less likely to occur as long as sufficient aromatics are present. Reaction rates and thermodynamic considerations also favor direct olefin-aromatic reactions. Whatever the involved mechanisms are, however, a range of alkylaromatic products can be expected with varying carbon numbers.

The objective normally will be to produce fuel products having a carbon number no higher than 14 and preferably not above 12 since the most valuable gasoline fuel hydrocarbons are at C₇-C₁₀ from the viewpoint of volatility including RVP and engine operation at varying conditions. Di-and tri-alkylation is therefore preferred since with the usual C₂, C₃ and C₄ olefins and a predominance of benzene in the aromatic feed, alkylaromatic products with carbon numbers from about 10 to 14 are readily achievable. Depending on the feed composition, operating conditions and type of unit, the product slate may be varied with optimum conditions for any given product distribution being determined empirically.

Process Parameters

The present process is notable for its capability of being operated at low temperatures and under moderate pressures. In general terms, the temperature will be from about 120° to 350° C. (about 250 to 660° F.) and in most cases between 150° and 250° C. (about 300 to 480° F). Temperatures of 170° to 180° C. (340° to 355° F.) will normally be found optimum for feeds comprising butene while higher temperatures will normally be appropriate for feeds with significant amounts of propene. Ethylene will require higher temperature operation to ensure satisfactory ethylene conversion. Pressures will normally be dependent on unit constraints but usually will not exceed about 10,000 kPag (about 1450 psig) with low to moderate pressures, normally not above 7,000 kPag (about 1,000 psig) being favored from equipment and operating considerations although higher pressures are not unfavorable in view of the volume change in the reaction; in most cases, the pressure will be in the range of 2000 to 5500 kPag (about 290 to 800 psig) in order to make use of existing equipment. Space velocities can be quite high, giving good catalyst utilization. Space velocities are normally in the range of 0.5 to 5 hr⁻¹ WHSV for the olefin feed, in most cases, 1 to 2 hr⁻¹ WHSV. Optimum conditions may be determined empirically, depending on feed composition, catalyst aging and unit constraints.

Two factors affecting choice of temperature will be the feed composition and the presence of impurities, principally in the olefin feed stream. As noted above, ethylene is less reactive than propylene and for this reason, ethylene containing feeds will require higher temperatures than feeds from which this component is absent, assuming of course that high olefin conversion is desired. From this point of view, reaction temperatures at the higher end of the range, i.e. above 180° C. or higher, e.g. 200° or 220° C. or higher, will be preferred for ethylene-containing feeds. Sulfur will commonly be present in the olefin feeds from the FCC unit in the form of various sulfur-containing compounds e.g. mercaptans, and since sulfur acts as a catalyst poison at relatively low reaction temperatures, typically about 120° C., but has relatively little effect at higher temperatures about 180° C. or higher, e.g. 200° C., 220° C., the potential for sulfur compounds being present may dictate a preferred temperature regime above about 150° C., with temperatures above 180° C. or higher being preferred, e.g. 200° or 220° C. or higher. Typically, the sulfur content will be above 1 ppmw sulfur and in most cases above 5 ppmw sulfur; it has been found that with a reaction temperature above about 180-220° C., sulfur levels of 10 ppmw can be tolerated with no catalyst aging, indicating that sulfur levels of 10 ppmw and higher can be accepted in normal operation.

Operation may take place under vapor phase, liquid phase or supercritical phase conditions (reactor inlet). Frequently, mixed phase conditions will prevail, depending on the feed composition and the conditions used. At the reactor outlet, liquid phase will prevail under normal conditions with the product including significant proportions of C₈, C₁₀ and higher hydrocarbons. With significant amounts of ethylene (FCC Off Gas) in the olefin feed, operation will commence (reactor inlet) in the vapor phase or under mixed phase conditions and when higher olefins including propylene and butene are present, operation may frequently commence in the supercritical phase. Vapor phase and liquid phase processes with preferred process configurations and process conditions are disclosed in co-pending, concurrently filed patent applications U.S. Ser. Nos. (claming priority from applications Ser. Nos. 60/656,946 and 60/656,945, entitled “Liquid Phase Aromatics Alkylation Process” and “Vapor Phase Aromatics Alkylation Process” to which reference is made for a description of these processes.

The ratio between the olefin and aromatic feed components is normally chosen to achieve the desired process objective, be it benzene reduction, olefin conversion or a number of objectives. If benzene reduction is the primary objective, a relatively low aromatics:olefin ratio is desirable in order to favor aromatics alkylation using the excess olefins. In this case, it is preferred that the ratio of aromatics to olefins should not exceed 1:1 by weight. Using ratios below 1 in this way will, besides decreasing benzene in the product, limit conversion and increase the extent of di-alkylation; conversely, using higher ratios above 1:1, for example, 1.5:1 (aromatic:olefin, by weight) will increase conversion and the benzene in the product but reduce di-alkylation. Optimal conditions may therefore be determined empirically depending on feed composition, available feed rates, product objectives and unit type.

By appropriate adjustment of the reaction conditions, the product distribution may be modified: shorter feed/catalyst contact times tend to a product distribution with lower molecular weight oligomers while relatively longer contact times lead to higher molecular weight (higher boiling products). So, by increasing feed/catalyst contact time, it is possible to produce products in the middle distillate boiling range, for example, an aromatic road diesel as well as kerojet blend stocks. Overall feed/catalyst contact time may be secured by operating at low space velocity or by increasing the recycle ratio to the reactor.

### EXAMPLE 1

An aromatic feed was alkylated in a fixed-bed reactor at 1725 kPag (250 psig) and temperatures varying from 180 to 330° C. (356 to 625° F.) with an olefin co-feed. The aromatic feed was either benzene or a reformate heart cut fraction having the composition shown in Table 4 below.

TABLE 4
Reformate Composition, wt. pct.

| | |
|---|---|
| C5 | 8.744 |
| C6 | 29.000 |
| Benzene | 24.157 |
| C7 | 11.734 |
| Toluene | 25.844 |
| C8 | 0.458 |
| Total | 99.937 |

The olefin feed was either chemical grade ethylene or propylene, mixed with nitrogen and hydrogen when simulating FCC Off Gas. The unit was started-up on chemical grade benzene (BZ) and ethylene only. Propylene was added at 2.5 days on 5 stream. Nitrogen and hydrogen diluents were added at 7 days to simulate FCC-Off-Gas. Propylene was removed at 15 days and added back again at 18 days to evaluate ethylene conversion in the absence of propylene.

Changes in feed composition and temperature were made during the run as indicated below.

| Days on Stream | Action | Inlet Temp. (° C.) |
|---|---|---|
| 0 (Startup) | Feed benzene (BZ) only | 180 |
| 2.5 | Feed BZ+C2═+C3═ | 180 |
| 4.0 | Change feed to Reformate | 180 |
| 7.0 | Add N2 and H2 diluents | 180 |
| 9.0 | Increase temp. | 204 |
| 10.0 | Increase temp. | 232 |
| 12.0 | Increase temp. | 260 |
| 13.0 | Increase temp. | 288 |
| 15.0 | C3═ off | 288 |
| 16.0 | Increase temp. | 315 |
| 17.0 | Increase temp. | 330 |
| 18.0 | C3═ on | 330 |
| 20.0 | Decrease temp. | 288 |

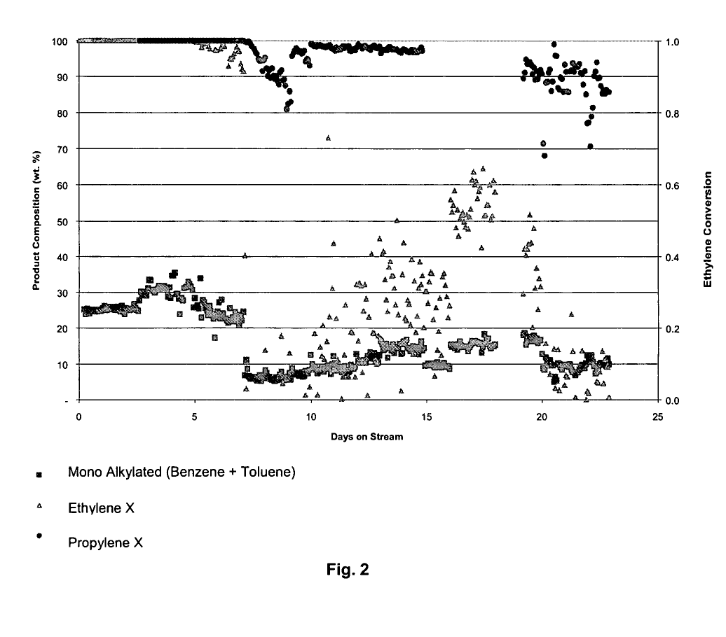

The results are shown in FIG. 2 and demonstrate high propylene conversion over MCM-22 in the vapor phase environment.

1. A method for the conversion of an SPA olefin oligomerization process unit to a process unit for producing a gasoline product of high octane rating and producing the gasoline in the unit, the method comprising converting an SPA olefin oligomerization process unit which includes a reactor in which light olefin feed is oligomerized to form gasoline boiling range hydrocarbon product, by adapting the unit to operation with a molecular sieve based olefin oligomerization catalyst, comprising the step of withdrawing solid phosphoric acid [SPA] catalyst from the unit and loading an olefin condensation catalyst comprising an MWW zeolite material into the reactor of the process unit and contacting a light olefin feed stream comprising C₂ to C₄ olefins and an aromatic, benzene-containing co-feed with the catalyst to react the olefins with benzene in the aromatic co-feed in an aromatics alkylation process.

2. A method according to claim 1 in which the MWW zeolite material comprises a member of the MCM-22 family of zeolites.

3. A method according to claim 2 in which the catalyst comprises a regenerated MCM-22 catalyst.

4. A method according to claim 1 in which the catalyst comprises a self-bound MCM-22 catalyst.

5. A method according to claim 1 in which the olefinic feed comprises a mixed light olefinic feed containing at least two olefins selected from ethylene, propene, butene, in which the alkylation process is operated at a temperature from 150 to 350° C. and a pressure of not greater than 7,000 kPag.

6. A method according to claim 1 in which the olefin feed is processed with the aromatic co-feed over the condensation catalyst for a cycle duration between successive regenerations of not less than six months.

7. A method according to claim 1 in which the aromatic co-feed contains from 5 to 60 weight percent benzene.

8. A method according to claim 1 in which the weight ratio of the aromatic co-feed to the olefin co-feed is less than 1:1.

9. A method according to claim 1 in which the weight ratio of the aromatic co-feed to the olefin co-feed is from 1:1 to 2:1.

10. A method according to claim 1 in which the reaction is carried out in a chamber reactor comprising a plurality of sequential fixed beds of catalyst or a tubular reactor comprising parallel reactors of tubular configuration immersed in liquid coolant.

11. A process for the production of high octane, aromatic gasoline boiling range blend component including products boiling in the C₅-200° C. range by the alkylation of a benzene-containing aromatic feed with mixed light olefins in the C₂ - C₄ range produced by the catalytic cracking of a petroleum feedstock in a fluid catalytic cracking unit, the process comprising passing the olefinic feed with a benzene-containing co-feed to a fixed bed of an olefin condensation/alkylation catalyst comprising as the active catalytic component, an MWW zeolitic material at a temperature from 150° to 350° C., a pressure not greater than 7000 kpa, and an olefin space velocity of not more than 5 WHSV [hour⁻¹].

12. A process according to claim 11 in which the average branching of the C₅-200° C. product is at least 1.8 [ME/C8].

13. A process according to claim 12 in which the average branching of the C₅-200° C. fraction is at least 2.25 [ME/C12].

14. A process according to claim 11 in which the feed comprises ethylene or propylene.

15. A process according to claim 11 in which the feed includes sulfur compounds and the reaction temperature is at least 180° C.

16. A process according to claim 11 in which the aromatic co-feed comprises from 5 to 60 vol. percent benzene.

17. A process according to claim 11 in which the octene components of the C5-200° C. product comprise at least 85 weight percent di-branched C8 hydrocarbons.

18. A process according to claim 16 in which the octene components of the C5-200° C. fraction comprises at least 88 to 96 weight percent di-branched C8 hydrocarbons.

19. A method for the conversion of an solid phosphoric acid [SPA] olefin oligomerization process unit which includes a reactor in which light olefin feed is oligomerized to form a gasoline boiling range hydrocarbon fuel product, which conversion method converts the SPA unit to operation with a molecular sieve based catalyst by withdrawing solid phosphoric acid catalyst from the unit, loading an aromatic alkylation catalyst comprising as the active catalytic component, an MWW zeolite material, into the reactor of the process unit to provide a fixed bed of the catalyst and producing a high octane rating, gasoline boiling range, gasoline blend component containing alkylaromatics by the catalytic alkylation of single ring aromatics in a light aromatic feedstock comprising benzene with light olefins in the C₂ - C₄ range in an olefinic feedstock produced by the catalytic cracking of a petroleum feedstock in a fluid catalytic cracking unit, by passing the olefinic and aromatic feedstocks to the fixed bed of catalyst in the reactor, at a temperature from 150 to 350° C., a pressure not greater than 7000 kPag, and an olefin space velocity of not more than 5 WHSV [hour⁻¹] and an aromatic:olefin weight ratio of not more than 2:1 to alkylate single ring aromatics in the aromatic feedstock.

20. A method according to claim 19 in which the aromatic feedstock comprises a reformate.
